(19) United States
(12) Patent Application Publication
(10) Pub. No.: US 2018/0190381 A1
(43) Pub. Date: Jul. 5, 2018

(54) Systems And Methods For Patient-Specific Prediction Of Drug Responses From Cell Line Genomics

(71) Applicant: Nantomics, LLC, Culver City, CA (US)

(72) Inventor: Christopher Szeto, Scotts Valley, CA (US)

(21) Appl. No.: 15/736,490

(22) PCT Filed: Jun. 15, 2016
(86) PCT No.: PCT/US2016/037641

§ 371 Date: Dec. 14, 2017

Related U.S. Application Data
(60) Provisional application No. 62/175,940, filed on Jun. 15, 2015.

(51) Int. Cl. G16H 50/20; G16H 50/50; G16H 20/10; G06N 99/00; G06F 19/24; G06F 19/28; G06F 19/18

(52) U.S. Cl. CPC G16H 50/20; G16H 50/50; G16H 20/10; G06F 19/18; G06F 19/24; G06F 19/28; G06N 99/005

(57) ABSTRACT

Contemplated systems and methods use a priori known cell line genomics and drug-response data to build a library of response predictors across multiple and distinct cell types and drugs. Statistical analysis of selected response predictors using actual patient data is then employed to identify a response predictor that has significant gain in prediction power, and the drug associated with the identified response predictor is then selected for treatment where the response predictor indicated sensitivity to the drug.

This application claims priority to U.S. provisional application No. 62/175,940, filed Jun. 15, 2015, which is incorporated herein by reference.

### FIELD OF THE INVENTION

The field of the invention is systems and methods of predicting drug responses using omics information.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

Various systems and methods of computational modeling of pathways are known in the art. For example, some algorithms (e.g., GSEA, SPIA, and PathOlogist) are capable of successfully identifying altered pathways of interest using pathways curated from literature. Still further tools have constructed causal graphs from curated interactions in literature and used these graphs to explain expression profiles. Algorithms such as ARACNE, MINDy and CONEXIC take in gene transcriptional information (and copy-number, in the case of CONEXIC) to so identify likely transcriptional drivers across a set of cancer samples. However, these tools do not attempt to group different drivers into functional networks identifying singular targets of interest. Some newer pathway algorithms such as NetBox and Mutual Exclusivity Modules in Cancer (MEMo) attempt to solve the problem of data integration in cancer to thereby identify networks across multiple data types that are key to the oncogenic potential of samples.

While such tools allow for at least some limited integration across pathways to find a network, they generally fail to provide regulatory information and association of such regulatory information with one or more effects in the relevant pathways or network of pathways. In an attempt to improve performance, GIENA looks for dysregulated gene interactions within a single biological pathway but does not take into account the topology of the pathway or prior knowledge about the direction or nature of the interactions. Moreover, due to the relative incomplete nature of these modeling systems, predictive analysis is often impossible, especially where interactions of multiple pathways and/or pathway elements are under investigation.

More recently, improved systems and methods have been described to obtain in silico pathway models of in vivo pathways, and exemplary systems and methods are described in WO 2011/139345 and WO 2013/062505. Further refinement of such models was provided in WO 2014/059036 (collectively referred to herein as “PARADIGM”) disclosing methods to help identify cross-correlations among different pathway elements and pathways. While such models provide valuable insights, for example, into interconnectivities of various signaling pathways and flow of signals through various pathways, numerous aspects of using such modeling have not been appreciated or even recognized.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Still further progress has been made using insights form PARADIGM as is described in WO 2014/193982. Here, multiple models are obtained from a machine learning system that receives multiple distinct data sets and identifies a determinant pathway element in the distinct data sets that is associated with a status (e.g., sensitive or resistant) of a treatment parameter (e.g., treatment with a drug) of the diseased cells. Such system advantageously provides insight into potential treatment modalities. However, the very large number of potentially valid models obtained from the machine learning system will render simple forecast of treatment outcome difficult.

On the other hand, as described in US 2004/0193019, discriminant analysis-based pattern recognition is disclosed to generate a model that correlates certain biological profile information with treatment outcome information. The prediction model is then used to rank possible responses to treatment. While such methods may help assess likely outcomes based on patient-specific profile information, analysis is typically biased by the parameters used in the discriminant analysis. Moreover, such analysis only takes into account historical data of corresponding drugs and disease conditions and so limits discovery of drugs known to be effective only in other non-related disease conditions. In addition, availability of the historical data of corresponding drugs and disease conditions tends to further limit usefulness of such methods.

Thus, even though various systems and methods for prediction of drug response are known in the art, there remains a need for a system and method that allows for simple and robust treatment prediction for a drug with high confidence, and that allows identification of a suitable drug in an agnostic manner.

### SUMMARY OF THE INVENTION

The inventive subject matter is directed to various devices, systems, and methods in which multiple a priori known cell line genomics and drug-response data are used to build a large number of response (therapy outcome) predictors that are then tested with actual patient data in a statistically controlled manner to identify a drug for treatment of the patient. Viewed from a different perspective, the inventors have discovered that matching a patient's pathway model with a response predictor that has a high gain of prediction score will readily identify one or more drugs for which treatment success or failure can be predicted at a desirably high confidence. Moreover, contemplated systems and methods also allow discovery of a drug for treatment in a disease for which the drug has previously not been known as therapeutically effective.

In one aspect of the inventive subject matter, the inventors contemplate various systems, methods, and non-transient computer readable media containing program instructions for identifying a drug for treatment of a cancer in a patient. In most preferred aspects, a machine learning system is informationally coupled to an analysis engine, and the machine learning system is used to calculate a first response predictor for a first cell with respect to a response of the first cell to a first drug, wherein the first response predictor is calculated using training data that include a pathway model of the first cell and a known response of the first cell to the first drug. The machine learning system is further used to calculate a second response predictor for a second cell with respect to response of the second cell to a second drug, wherein the second response predictor is calculated using training data comprising a pathway model of the second cell and a known response of the second cell to the second drug. The analysis engine then calculates respective null models for the first and second response predictors, and further calculates respective treatment responses according to the first and second response predictors using a pathway model of the patient. Moreover, the analysis engine then ranks the respective calculated treatment responses using the respective null models, and the ranking is used to identify the drug.

Contemplated machine learning system may uses various classifiers, including linear kernel support vector machines, first or second order polynomial kernel support vector machines, ridge regression, elastic net algorithms, sequential minimal optimization algorithms, random forest algorithms, naive Bayes algorithms, and/or a NMF predictor algorithm. Moreover, it should be noted that the machine learning system will preferably use multiple and distinct classifiers to generate respective multiple and distinct first response predictors and respective multiple and distinct second response predictors.

While not limiting to the inventive subject matter, it is contemplated that the first and second cells are distinct cancer cells, and/or that the first and second drugs are distinct drugs. With respect to the pathway model it is contemplated that suitable models include factor-graph-based models (e.g., PARADIGM), collections of expression data, and/or collections of copy numbers, which may be further processed in factor-graph-based models.

Most typically, the known response is treatment sensitivity or treatment resistance to the drug, and null models are calculated using training data other than the training data used for calculation of the first and second response predictors. It is further preferred that the first and second response predictors are fully trained models, and that the step of ranking uses accuracy gain of the calculated treatment responses relative to the corresponding null models.

In another aspect of the inventive subject matter, the inventors contemplate various systems, methods, and non-transient computer readable media containing program instructions for a method of identifying a drug for treatment of a cancer in a patient. Here, a response predictor database is coupled to an analysis engine, and the response predictor database provides a plurality of response predictors to the analysis engine. Each of the response predictors is preferably calculated by a machine learning system that uses training data comprising a pathway model of a cell and a known response of the cell to a drug. The analysis engine then uses a plurality of randomly selected pathway models to generate respective null models for the plurality of response predictors, and further uses a patient pathway model to generate respective test models for the plurality of response predictors. Most typically, the analysis engine then ranks the respective test models by their respective gain in prediction score relative to their corresponding null models and identifies a drug based on a rank in the ranked test model.

Most typically, but not necessarily, the plurality of response predictors are fully trained models and/or high accuracy gain models. As noted above, it is contemplated that the machine learning system may use various classifiers, including linear kernel support vector machines, first or second order polynomial kernel support vector machines, ridge regression, elastic net algorithms, sequential minimal optimization algorithms, random forest algorithms, naive Bayes algorithms, and NMF predictor algorithms.

Most typically, contemplated pathway models include factor-graph-based models (and especially PARADIGM), collection of expression data, and/or or a collection of copy numbers. It is further contemplated that the pathway model may be generated from cancer and matched normal tissue data. Where desired, the randomly selected pathway models are generated from respective different cells, and a plurality of randomly selected non-patient pathway models may be used to generate respective patient null models for the plurality of response predictors (which may then be compared with the null models).

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIGS. 1A-1C schematically illustrate exemplary aspects of response predictors.

FIGS. 2A-2B exemplarily and schematically illustrate a process according to the inventive subject matter.

FIG. 3 exemplarily illustrates a ranked listing of calculated treatment responses/test models in which responses/models with higher accuracy gain over null models are placed to the left of those with lower accuracy gain. The calculated treatment response/test model at the far left predicted sensitivity of the patient to dasatinib with the highest accuracy gain.

FIG. 4 depicts exemplary results of accuracy gains for different calculations using different pathway models.

FIG. 5 is an exemplary representation of dasatinib sensitivity sorted by cell line type.

FIG. 6 is an exemplary representation of dasatinib sensitivity sorted by human TCGA tumor type.

### DETAILED DESCRIPTION

An overwhelming amount of machine learned predictive models can be prepared that allow calculation of a prediction (e.g., sensitivity) score on the basis of various omics datasets and/or pathway models prepared from omics datasets. Unfortunately, all of these models have various inherent biases, for example, due to underlying mathematical assumptions in machine learning and pathway construction, use of specific cell cultures or biopsy samples to obtain the omics data, the drug used with the cell cultures or biopsy samples, etc. Nevertheless, all of these models are based on actual cell biological processes and therefore provide at least potentially valuable insights. However, none of the diverse models provides any guidance as to which model will provide a match to a patient omics sample or pathway model that would predict whether or not a particular drug is likely to have a desired treatment outcome in the patient.

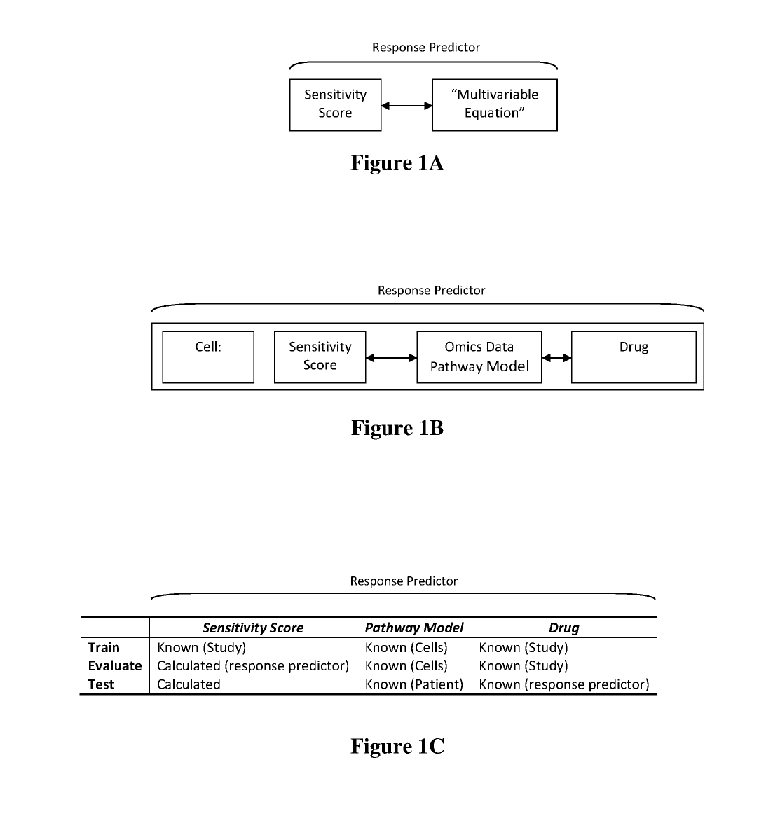

The inventors have now discovered systems and methods for matching actual patient data, and particularly pathway models from data of a patient, with a response predictor that has a desirably high gain of accuracy over a corresponding null model, which in turn allows identification of a drug that is predicted with high probability to have a therapeutic effect. In that context, as simplified in FIG. 1A, an exemplary response predictor (predictive model) can be viewed as multivariable equation obtained from a machine learning algorithm that will give a sensitivity or prediction score. More particularly, and as further exemplarily illustrated in FIG. 1B, a response predictor is generated using a machine learning algorithm that uses omics data and/or pathway models generated from a cell culture or tissue exposed to a drug. As is indicated in FIG. 1B, cells or tissue are exposed to a drug and sensitivity is observed (e.g., quantified as IC₅₀, EC₅₀, etc., or qualitatively assessed as sensitive or resistant), most typically in comparison with a negative or otherwise contrasting control (e.g., without drug or with different cell type). Omics data and/or pathway models from the cells/tissue are then used in a machine learning algorithm together with the observed factors as training data to so arrive at a response predictor. Of course, it should be appreciated that the same omics data and/or pathway models and observed factors can be used as training data in more than one machine learning algorithm, and it should be appreciated that all known machine learning algorithms are deemed suitable for use herein. Consequently, it should be appreciated that one set of in vitro experiments can provide a multiplicity of trained models (i.e., response predictors generated by respective machine learning algorithms). As is also well known in the art, available data may be split into a training set and evaluation set to obtain trained models, or all data can be used to get a fully trained model. Viewed from a different perspective, and as schematically shown in FIG. 1C, a response predictor can be generated using machine learning algorithms using training data where sensitivity of a cell or tissue to a drug is known, where the drug is known, and where the omics data and/or pathway model is readily obtained from the cells or tissue. So generated trained models can be validated using evaluation data which can be from the same dataset as the training data, and as before, the sensitivity of a cell or tissue to the drug is known, the drug is known, and the omics data and/or pathway model are readily obtained from the cells or tissue. Thus, it should be appreciated that numerous in vitro tests will form the basis for a large variety of response predictors that can then be used for calculation with a patient's omics data or pathway models. Using the patient omics data or pathway models in conjunction with the response predictors will then provide a predicted response score (predicted treatment outcome, or predicted sensitivity) for a drug.

Most advantageously, it should be recognized that contemplated systems and methods take advantage of the growing number of omics information associated with drugs and cells or tissue types. Using such information, a vast number of individual response predictors can be prepared, and it should be further recognized that the collection of response predictors need not even be limited to a specific cancer type and/or therapeutic drug. For example, as is further explained in more detail below, the inventors obtained different omics data sets from publically available sources (e.g., CCLE expression, CCLE copy number, sanger expression, sanger copy number) as pathway model omics data, and also used the same omics data in a factor-graph-based pathway model (here: PARADIGM) to end up with 10 different input data collections for which 139 different drugs were reported. These pathway models and known drug responses were then subjected to 13 different machine learning algorithms (Linear kernel SVM, First order polynomial kernel SVM, Second order polynomial kernel SVM, Ridge regression, Lasso, Elastic net, Sequential minimal optimization, Random forest, J48 trees, Naive bayes, JRip rules, HyperPipes, and NMFpredictor) resulting in a total of 176,112 response predictors.

In this context it must be noted that each type of response predictor includes inherent biases or assumptions, which may influence how a resulting response predictor would operate relative to other types of response predictors, even when trained on identical data. Accordingly, different response predictors will produce different predictions/accuracy gains when using the same training data set. Heretofore, in an attempt to improve prediction outcome, single machine learning algorithms were optimized to increase correct prediction on the same data set. However, due to inherent bias of the algorithms, such optimization will not necessarily increase accuracy (i.e., accurate prediction capability against ‘coin flip’) in predictability. Such bias can be overcome by training numerous diverse response predictors with different underlying principles and classifiers on disease-specific data sets with associated metadata and by selecting from the so trained response predictors those with desirable prediction power over the corresponding null model.

Of course, it should be appreciated that the above is only an exemplary and relatively limited set of data, and that numerous additional data (e.g., in vitro data, clinical trial data, research data, treatment data, etc.) can be employed, each in combination with their respective drugs, and each calculated with different machine learning algorithms to so arrive at very large numbers (e.g., between 100,000-500,000, or between 500,000 and 1,000,000, or between 1,000,000 and 5,000,000, or between 5,000,000 and 10,000,000, and even more) of individual response predictors. As should be evident, such calculations well exceed multiple lifetimes of a human without computing infrastructure.

As should also be readily appreciated, even with computing infrastructure, such large data quantities would require immense computational effort where an actual dataset (omics data or pathway model) of a patient should be aligned with a dataset of cell or tissue culture. The inventors have now discovered that even massive collections of response predictors can be effectively and expeditiously analyzed in a conceptually simple manner by calculating two predicted responses for a single response predictor, using a simulated null set and an actual patient dataset (omics data or pathway model). Differences between the predicted responses are then used to evaluate the performance of the single response predictor. In that manner, only relatively simple calculations are required and can be performed in a comparably small amount of time as the response predictors are relatively simple (see FIGS. 1A and 1B).

Consequently, it should be noted that the inventive subject matter presented herein enables construction or configuration of a computing device(s) to operate on vast quantities of digital data, beyond the capabilities of a human. Although the digital data can represent machine-trained computer models of omics data and treatment outcomes, it should be appreciated that the digital data is a representation of one or more digital models of such real-world items, not the actual items. Rather, by properly configuring or programming the devices as disclosed herein, through the instantiation of such digital models in the memory of the computing devices, the computing devices are able to manage the digital data or models in a manner that would be beyond the capability of a human. Furthermore, the computing devices lack a priori capabilities without such configuration. In addition, it should be appreciated that the present inventive subject matter significantly improves/alleviates problems inherent to computational analysis of complex omics calculations.

Viewed from a different perspective, it should be appreciated that the present systems and methods in computer technology is being used to solve a problem inherent in computing models for omics data. Thus, without computers, the problem, and thus the present inventive subject matter, would not exist. More specifically, systems and methods presented herein result in one or more response predictors models having greater accuracy gain than others, which results in less latency in generating predictive results based on actual patient data.

It should be noted that any language directed to a computer, analysis engine, or machine learning system should be read to include any suitable combination of computing devices, including servers, interfaces, systems, databases, agents, peers, engines, controllers, modules, or other types of computing devices operating individually or collectively. One should appreciate the computing devices comprise a processor configured to execute software instructions stored on a tangible, non-transitory computer readable storage medium (e.g., hard drive, FPGA, PLA, solid state drive, RAM, flash, ROM, etc.). The software instructions configure or otherwise program the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In some embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network, circuit switched network, and/or cell switched network.

As used in the description herein and throughout the claims that follow, when a system, engine, server, device, module, or other computing element is described as configured to perform or execute functions on data in a memory, the meaning of “configured to” or “programmed to” is defined as one or more processors or cores of the computing element being programmed by a set of software instructions stored in the memory of the computing element to execute the set of functions or operate on target data or data objects stored in the memory.

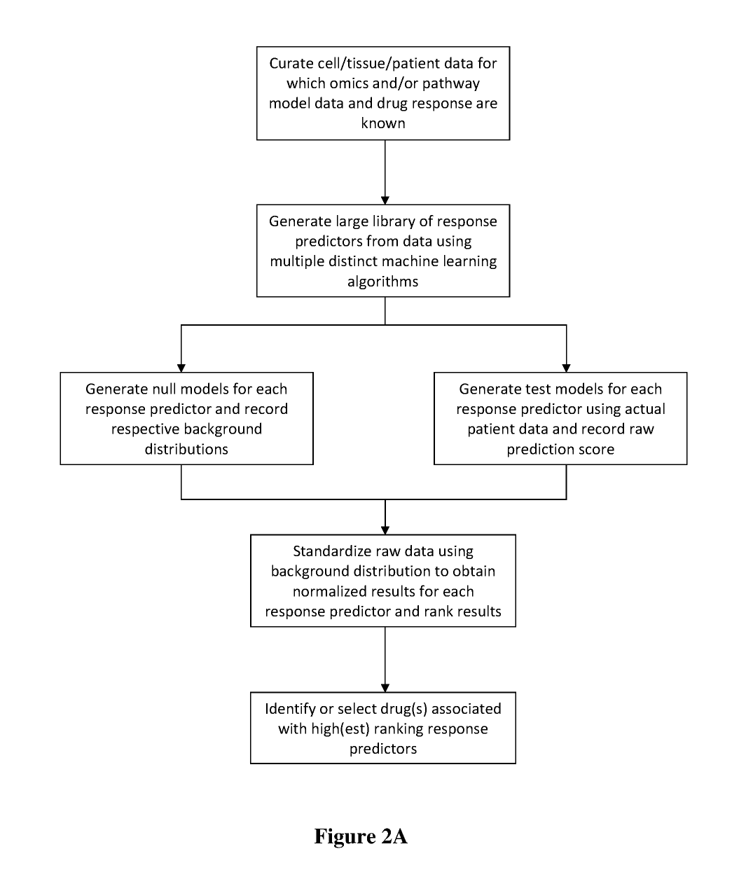

The flow chart of FIG. 2A exemplarily illustrates the above, and FIG. 2B gives a more detailed overview of the chart of FIG. 2A. Here, numerous distinct known cell lines (e.g., liver cells and pancreatic cells) were tested with different drugs (e.g., D₁, D₂, . . . Dₙ) for which sensitivity or resistance to the drugs was known or established, and for each of the cell cultures, omics analysis and pathway modeling was performed to so arrive at corresponding pathway models (e.g., L-PM_{A1} for liver cells of a particular cell type (A) treated with a particular drug (D₁), etc.). Using this information (e.g., drug response and pathway model for the specific cell, typically in conjunction with negative control and/or other parameter), a particular response predictor (e.g., RP-L_{A1}) can be calculated using a specific machine learning algorithm. As noted above, multiple different drugs, omics datasets, pathway modeling, and cell types can be used with multiple different machine learning algorithms, which exponentially increases the number of available response predictors (not shown in the example of FIG. 2B). The so generated response predictors are then assembled into a response predictor database.

Once the response predictors are created, prediction quality may be assessed, and most preferably response predictors are retained that have a prediction power that exceeds random selection. Viewed from a different perspective, models may be assessed on their gain in accuracy. There are numerous manners of assessing accuracy, and the particular choice may depend at least in part on the algorithm used. For example, suitable metrics include an accuracy value, an accuracy gain, a performance metric, or other measure of the corresponding model. Additional example metrics include an area under curve metric, an R², a p-value metric, a silhouette coefficient, a confusion matrix, or other metric that relates to the nature of the response predictor. Depending on the number of response predictors or accuracy distribution, it should be appreciated that the response predictor used for prediction may be selected as the top model (having highest accuracy gain, or highest accuracy score, etc.), or as being in the top n-tile (tertile, quartile, quintile, etc.), or as being in the top n % of all models (top 5%, top 10%, etc.). For example, high accuracy gain models will typically be in the top quartile of accuracy gain.

This database is then used for statistical selection of matches with a high prediction score for actual patient data using null models for each of the response predictors in the database. More specifically, null models are calculated for each of the response predictors using a moderate number (e.g., 100-500, or 500 to 1,000, or 1,000 to 10,000) of randomly chosen datasets (e.g., pathway models or omics data used in the calculation of the response predictors, but not used in calculation of the response predictor for which the null model is created). As can be expected, the null models will provide a background signal distribution (e.g., mean and standard deviation) for unrelated or poorly-matched pathway models or omics data. Then, actual patient data are used in the response predictors of the database to prepare prediction scores (sensitivity or resistance scores) so that two results are available for each response predictor of the database. Once more such calculation is rapid due to the simplified data structure of the response predictors and will not require a machine learning process in which patient data are attempted to conform to in vitro model data as would be commonly done.

In situations where one response predictor predicts a high prediction score (e.g., high level of sensitivity or resistance) for the actual patient data and an average prediction score for the randomly chosen datasets (background signal), a high score is noted as the raw score that is then adjusted using the background signal distribution to so arrive at a standardized score. It should be appreciated that this standardized score characterizes the conformance of the patient data set with the performance of the response predictor as originally calculated with the drug of a particular cell or tissue. Thus, a higher prediction score for a response predictor using a patient dataset (pathway model or omics data) indicates that the patient's response to treatment with the drug used in the response predictor may also be accurately predicted. Viewed from a different perspective, where the original patient dataset is more similar to the original dataset used in the calculation of a prediction model, a higher prediction score is observed (as the prediction model is optimized for predicting a response to a specific drug). FIG. 2 provides an exemplary comparison between null model and corresponding test model or Topmodel (model with highest accuracy gain among corresponding models), and the difference in raw score, and more preferably the difference in standardized score is then used for ranking. Top ranking response predictors and their associated drugs are identified, and the so identified drugs (marked with an asterisk or two asterisks) can then be suggested or used for treatment.

Based on omics and pathway data from patients diagnosed with glioblastoma and response predictors built from known data with different cell types and drugs and associated sensitivity to the drugs as shown in Table 1 below, dasatinib was identified as a drug suitable for the patients.

TABLE 1

| Types | | Number |
|---|---|---|
| Genomic datasets | CCLE expression | 10 (8320 samples) |
| | CCLE copy number | |
| | CCLE expression paradigm | |
| | CCLE copy number paradigm | |
| | CCLE expression & copy | |
| | number paradigm | |
| | sanger expression | |
| | sanger copy number | |
| | sanger expression paradigm | |
| | sanger copy number paradigm | |
| | sanger_expression & copy | |
| | number paradigm | |
| Drugs | 17-AAG | 139 |
| | 681640 | |
| | A-443654 | |
| | A-770041 | |
| | . . . | |
| | WZ-1-84 | |
| | XMD8-85 | |
| | Z-LLNIe-CHO | |
| | ZM-447439 | |
| Classifiers | Linear kernel SVM | 13 |
| | First order polynomial | |
| | kernel SVM | |
| | Second order polynomial | |
| | kernel SVM | |
| | Ridge regression | |
| | Lasso | |
| | Elastic net | |
| | Sequential minimal optimization | |
| | Random forest | |
| | J48 trees | |
| | Naive bayes | |
| | JRip rules | |
| | HyperPipes | |
| | NMFpredictor | |
| Feature selections | Four levels of variance filters | 4 |

Using the above, 29,352 fully trained drug response models were built, 146,760 additional evaluation models were built (at 5-fold CV), and 176,112 total models were analyzed. Genomic-scale data from patients were collected from individual cancer samples via microarray or sequencing technology. Several independent assays were performed on the same samples (e.g., both expression profiling and copy-number estimation) to evaluate what data type will provide best predictions. These data were integrated in a factor-graph-based model using PARADIGM. The most likely state for the pathway networks given the -omics data evidence is estimated, and reported as inferred pathway activities (pathway model). Thus, it should be especially appreciated that contemplated systems and methods are neither based on prediction optimization of a singular model nor based on identification of best correlations of selected omics parameters with a treatment prediction.

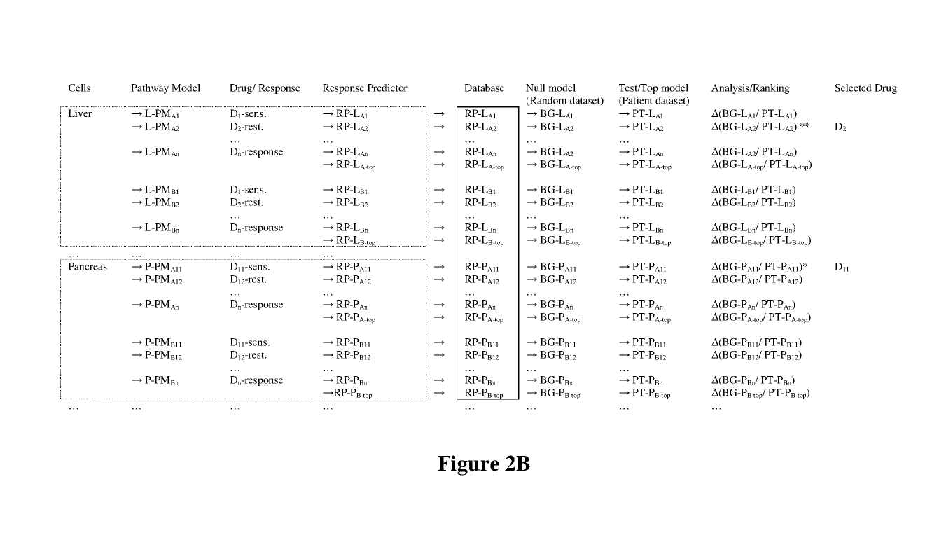

Using the so built response predictor database and patient data, null models were then calculated for each of the response predictors with 1,000 randomly selected datasets, and mean and standard deviation were recorded for each null model. Test models were then also calculated using patient datasets for each of the response predictors and the results standardized using the results from the respective null models. FIG. 3 exemplarily shows ranking of standardized scores. Here, each vertical line represents average, minimum, and maximum results for a number of response predictors grouped by a specific drug. As can be seen from FIG. 3, response predictors to the left are more consistently accurately predicted, and the most consistently predicted drug is dasatinib. Most notably, it should be appreciated that dasatinib was originally developed as an oral Bcr-Abl tyrosine kinase inhibitor (inhibits the “Philadelphia chromosome”) and was approved for first line use in patients with chronic myelogenous leukemia and Philadelphia chromosome-positive acute lymphoblastic leukemia. Thus, it should be appreciated that a response to a drug in a patient can be predicted on the basis of omics data/pathway models of the patient when used as input data to a collection of prediction models where each of the models was optimized to predict drug response as a function of a specific set of omics data/pathway models. Moreover, by comparing predicted results to a null model, statistically relevant predictions above background are reported. Additionally, to ensure that the patient data do not import an inherent bias, permutations can also be generated from the patient data that are then classified in a manner as described for the null models to ensure that the patient data and the null model are distributed similarly.

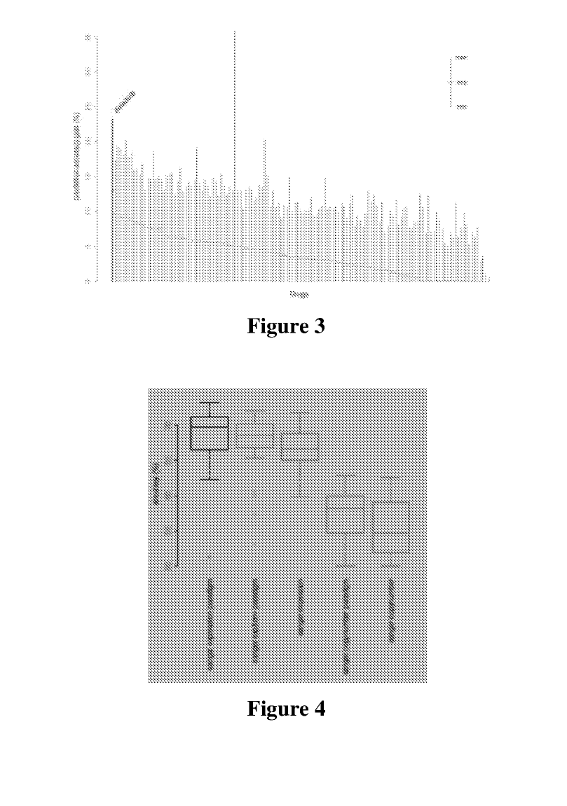

With respect to the omics data and pathway models suitable for use herein, it should be noted that all omics data and pathway models are deemed appropriate, and exemplary omics data include sequencing data, especially tumor versus normal data, such as whole genome sequencing data, exome sequencing date, etc. Moreover, suitable omics data also include transcriptomics data and proteomics data. Likewise, suitable pathway models include Gene Set Enrichment Analysis (GSEA, Broad Institute) based models, Signaling Pathway Impact Analysis (SPIA, Bioconductor) based models, and PathOlogist pathway models (NCBI) as well as factor-graph based models, and especially PARADIGM as described in WO2011/139345A2, WO2013/062505A1, and WO2014/059036, all incorporated by reference herein. FIG. 4 provides exemplary comparative results depicting average accuracy as a function of the type of omics data and pathway models. As can be clearly seen, the highest accuracy was achieved using Sanger expression data that were processed using PARADIGM to so obtain a pathway model. Similarly high accuracy was achieved using Sanger expression and copy number data, again processed using PARADIGM to so obtain the corresponding pathway model. Notably, Sanger expression data alone without pathway modeling also afforded relatively high, albeit somewhat lower, accuracy. Copy number omics data only, per se or processed using PARADIGM, ranked somewhat lower.

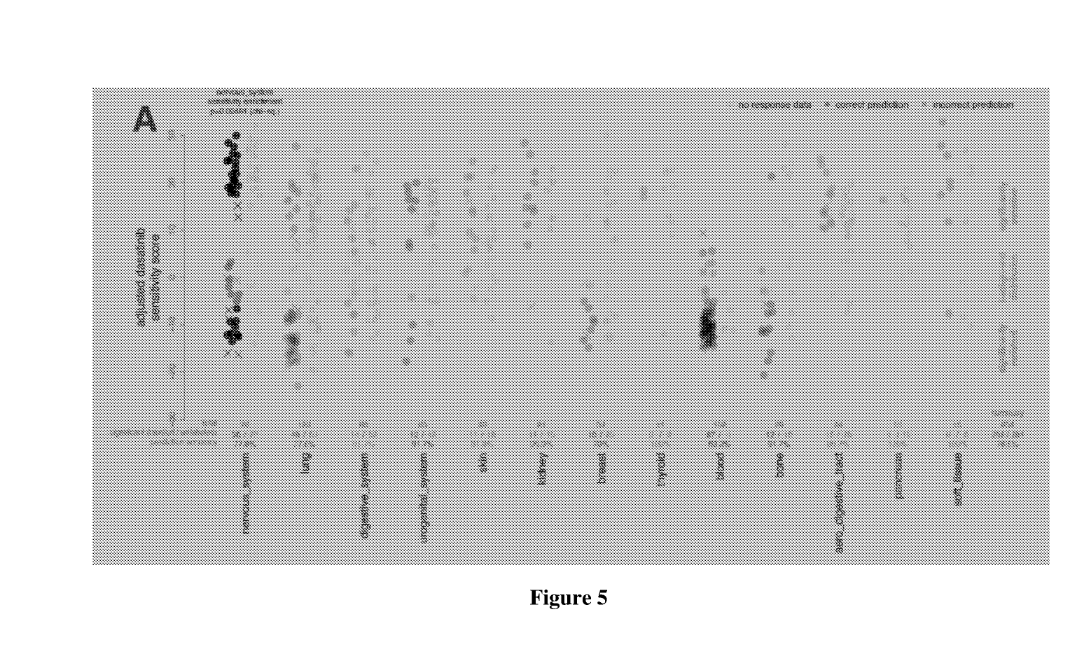

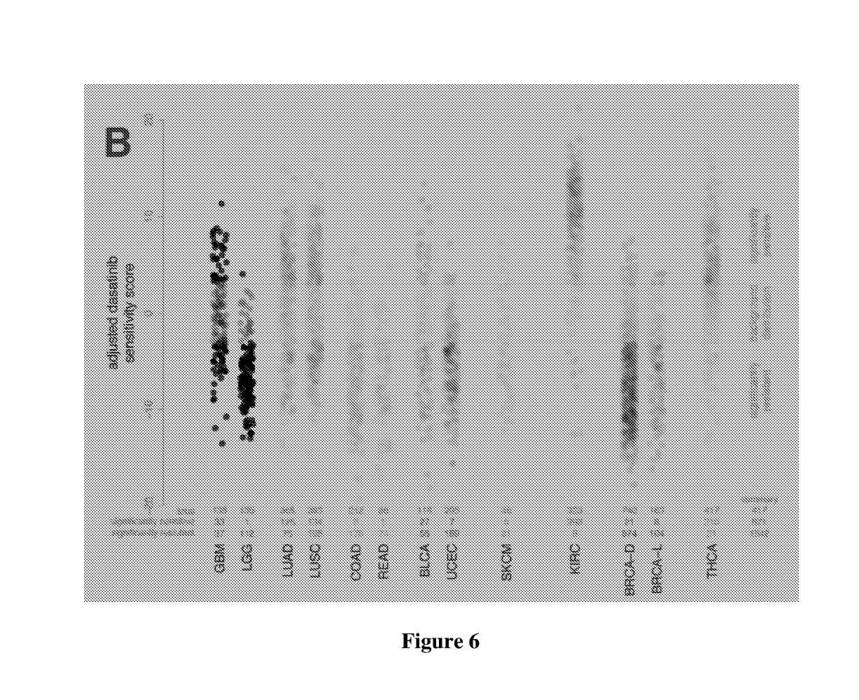

The accuracy of the so obtained predictions was cross checked using omics data and pathway models for cell lines, and the results are depicted in FIG. 5. Here, the adjusted sensitivity scores are plotted with solid circles indicating predictions for which sensitivity data were available, with empty circles indicating predictions for which sensitivity data were not available, and labeled with x for incorrect predictions. Notably, prediction accuracy for dasatinib in neural cell lines was 77.8%, which coincides with the prediction for glioblastoma patients. Equally notable is that dasatinib resistance can be accurately predicted as well as can be taken from FIG. 5. A similar cross check was performed using primary patient data from TCGA samples in tissues that correspond to the training cell line panel as can be seen from FIG. 6. Note that the tissue effects behave similarly between cell line and patient data. For example, similarly to neural system lines, GBM patient samples are predicted to contain responder and non-responder subsets. In addition, it should be noted that dasatinib may be an excellent alternate drug candidate for human renal clear cell carcinoma.

Further considerations suitable for use herein are disclosed in WO 2014/193982 and PCT/US16/13959, entitled “Ensemble-Based Research Recommendation Systems and Methods”, filed 19 Jan. 16, and incorporated by reference herein.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A method of identifying a drug for treatment of a cancer in a patient, comprising:
informationally coupling a machine learning system to an analysis engine;
using the machine learning system to calculate a first response predictor for a first cell with respect to a response of the first cell to a first drug;
wherein the first response predictor is calculated using training data that include a pathway model of the first cell and a known response of the first cell to the first drug;
using the machine learning system to calculate a second response predictor for a second cell with respect to response of the second cell to a second drug;
wherein the second response predictor is calculated using training data comprising a pathway model of the second cell and a known response of the second cell to the second drug;
calculating, by the analysis engine, respective null models for the first and second response predictors;
calculating, by the analysis engine, respective treatment responses according to the first and second response predictors using a pathway model of the patient, and ranking, by the analysis engine, the respective calculated treatment responses using the respective null models; and
using the ranking to identify the drug.

2. The method of claim 1 wherein the machine learning system uses a classifier selected form the group consisting of a linear kernel support vector machine, a first or second order polynomial kernel support vector machine, a ridge regression, an elastic net algorithm, a sequential minimal optimization algorithm, a random forest algorithm, a naive Bayes algorithm, and a NMF predictor algorithm.

3-11. (canceled)

12. The method of claim 1 wherein the machine learning system uses multiple and distinct classifiers to generate respective multiple and distinct first response predictors and respective multiple and distinct second response predictors.

13. The method of claim 1 wherein the first and second cells are distinct cancer cells.

14. The method of claim 1 wherein the first and second drugs are distinct drugs.

15. The method of claim 1 wherein the pathway model is a factor-graph-based model, a collection of expression data, or a collection of copy numbers.

16. The method of claim 15 wherein the factor-graph-based model is PARADIGM.

17. The method of claim 1 wherein the known response is treatment sensitivity to a drug or treatment resistance to the drug.

18. The method of claim 1 wherein the null models are calculated using training data other than the training data used for calculation of the first and second response predictors.

19. The method of claim 1 wherein the first and second response predictors are fully trained models.

20. The method of claim 1 wherein the step of ranking uses accuracy gain of the calculated treatment responses relative to the corresponding null models.

21. A method of identifying a drug for treatment of a cancer in a patient, comprising:
informationally coupling a response predictor database to an analysis engine;
providing, by the response predictor database, a plurality of response predictors to the analysis engine, wherein each of the response predictors is calculated by a machine learning system using training data comprising a pathway model of a cell and a known response of the cell to a drug;
using, by the analysis engine, a plurality of randomly selected pathway models to generate respective null models for the plurality of response predictors;
using, by the analysis engine, a patient pathway model to generate respective test models for the plurality of response predictors;
ranking, by the analysis engine, the respective test models by their respective gain in prediction score relative to their corresponding null models; and
identifying, by the analysis engine, a drug based on a rank in the ranked test model.

22. The method of claim 21 wherein the plurality of response predictors are fully trained models.

23-28. (canceled)

29. The method of claim 21 wherein the plurality of response predictors are high accuracy gain models.

30. The method of claim 21 wherein the machine learning system uses a classifier selected form the group consisting of a linear kernel support vector machine, a first or second order polynomial kernel support vector machine, a ridge regression, an elastic net algorithm, a sequential minimal optimization algorithm, a random forest algorithm, a naive Bayes algorithm, and a NMF predictor algorithm.

31. The method of claim 21 wherein the pathway model is a factor-graph-based model, a collection of expression data, or a collection of copy numbers.

32. The method of claim 21 wherein the pathway model is generated from cancer and matched normal tissue data.

33. The method of claim 21 wherein the randomly selected pathway models are generated from respective different cells.

34. The method of claim 21 further comprising a step of using, by the analysis engine, a plurality of randomly selected non-patient pathway models to generate respective patient null models for the plurality of response predictors, and comparing the patient null models with the null models.

35-102. (canceled)
